(19) United States
(12) Patent Application Publication
(10) Pub. No.: US 2026/0124152 A1
(43) Pub. Date: May 7, 2026

(54) METHOD OF PRODUCING ENGINEERED EXOSOME-MIMETIC VESICLES

(71) Applicant: Hong Kong Centre for Cerebro-Cardiovascular Health Engineering Limited, Pak Shek Kok (HK)

(72) Inventors: Peng Shi, Pak Shek Kok (HK); Xi Zhao, Pak Shek Kok (HK)

(21) Appl. No.: 19/366,939

(22) Filed: Oct. 23, 2025

Related U.S. Application Data
(60) Provisional application No. 63/716,310, filed on Nov. 5, 2024.

(51) Int. Cl. A61K 9/50; A61K 31/704; A61K 47/64; A61K 47/69; C12N 5/071

(52) U.S. Cl. CPC A61K 9/5068; A61K 31/704; A61K 47/6425; A61K 47/6911; C12N 5/0686; C12N 2510/00

(57) ABSTRACT

A method for producing one or more engineered exosome-mimetic vesicles for targeted drug delivery, including, engineering one or more eukaryotic cells to stably express a Chimeric Antigen Receptor (CAR) protein on their surface; incubating the one or more engineered eukaryotic cells with a therapeutic cargo; and subjecting the one or more incubated cells to a mechanical extrusion process to form nanoscale one or more engineered exosome-mimetic vesicle with the CAR protein displayed on their surface and the therapeutic cargo encapsulated within.

### TECHNICAL FIELD

The present disclosure relates to a method of producing one or more engineered exosome-mimetic vesicles, and an engineered exosome-mimetic vesicle.

### BACKGROUND

Immunotherapy represents a significant advancement in modern medicine, utilising the body's natural defence mechanisms to combat disease. These therapeutic approaches have shown considerable promise in treating various conditions, particularly in oncology, where they offer targeted intervention strategies that differ from traditional treatment modalities.

In the field of targeted immunotherapies researchers seek to harness the specificity of immune receptors and the biocompatibility of extracellular vesicles. However, current treatment modalities encounter challenges related to safety profiles, manufacturing complexity, and therapeutic accessibility that limit widespread clinical implementation.

The ongoing advancement of immunotherapy represents an important area of medical research with potential implications for improving patient outcomes across various disease contexts through enhanced treatment accessibility and improved clinical applications. Methods of improved targeted drug delivery are desired.

### SUMMARY OF THE INVENTION

The present disclosure pertains to the field of biotechnology and nanomedicine, specifically focusing on the development of biomimetic vesicle systems for targeted drug delivery in oncology. In particular, the present disclosure relates to a method of producing an engineered exosome. In a preferred embodiment, the present disclosure relates to a method of producing a Chimeric Antigen Receptor (CAR) exosome. In an example embodiment, the CAR-exosome has targeted drug delivery capabilities. In one example, there is disclosed an apparatus for one step mechanical extrusion of drug loaded CAR-exosomes.

The present invention provides a method of producing one or more engineered exosome-mimetic vesicles, and an engineered exosome-mimetic vesicle.

In accordance with a first aspect, the present disclosure provides a method for producing one or more engineered exosome-mimetic vesicles for targeted drug delivery, comprising:
engineering one or more eukaryotic cells to stably express a Chimeric Antigen Receptor (CAR) protein on a eukaryotic cell surface;
incubating the one or more engineered eukaryotic cells with a therapeutic cargo; and
subjecting the one or more incubated cells to a mechanical extrusion process to form nanoscale exosome-mimetic vesicles with the CAR protein displayed on the eukaryotic cell surface and the therapeutic cargo encapsulated within.

In one example, the eukaryotic cells are human embryonic kidney (HEK293) cells.

In one example, the CAR protein is a CD19-targeting CAR.

In one example, the therapeutic cargo is doxorubicin (DOX).

In an example embodiment, the mechanical extrusion process comprises sequentially extruding the cells through polycarbonate porous membranes having progressively smaller pore sizes.

The claimed method advantageously produces engineered exosome-mimetic vesicles (CAR-exosomes) which combine the targeting specificity of CAR-T cells with the high drug delivery capacity of exosomes. The method provides high production efficiency. The method is also advantageous because the drug e.g., DOX, is loaded into the exosome-mimetic vesicle during vesicle formation, compared to post isolation loading. This is advantageous because it preserves vesicle integrity and improves drug encapsulation efficiency.

In one example, the polycarbonate porous membranes comprise pore sizes of 5 μm, 0.8 μm, and 0.4 μm. Alternatively, the pore size may be 10 μm, 5 μm, 1 μm, 0.4 μm or 0.2 μm, or as in some examples, the porous membranes may include pores that are of a single or multiple (combination) of pore sizes, such as (10 μm, 5 μm, 1 μm, 0.4 μm), (5 μm, 1 μm, 0.4 μm, 0.2 μm), (5 μm, 0.8 μm, 0.2 μm) or any combination thereof. Such combination of membrane pores may be determined based on the cell type, operation method and specific application.

In one example, a first extrusion is performed through a polycarbonate porous membrane having a pore size of 5 μm, a second extrusion is performed through a polycarbonate porous membrane having a pore size of 0.8 μm, and a third extrusion is performed through a polycarbonate porous membrane having a pore size of 0.4 μm.

In one example, the step of engineering the human embryonic kidney cells (HEK293) comprising the step of performing plasmid transfection on the HEK293 cells to engineer the HK293 cells to express CD19-targeting CAR protein.

In one example, the cells were prepared as a suspension (3×10⁶ cells/ml)

In one example, the method comprising the step of isolating the engineered exosome-mimetic vesicles. The engineered exosome-mimetic vesicles may be isolated by an appropriate isolation process e.g., density gradient ultracentrifugation or tangential flow filtration or ultrafiltration or any other suitable isolation process.

In one example, the engineered exosomes comprised a peak size of 145 nm.

In one example, the engineered exosome is configured to achieve a cumulative release concentration of 5.57 μg/ml at 9 hours.

In one example, the cells are incubated with DOX for two hours.

In accordance with a further aspect, the present disclosure provides a method of producing chimeric antigen receptor-presenting exosome-mimetic vesicles loaded with a therapeutic agent, comprising:
stably transfecting human embryonic kidney (HEK293) cells with a nucleic acid encoding a CD19-targeting chimeric antigen receptor;
incubating the transfected HEK293 cells in a suspension comprising doxorubicin (DOX);
sequentially extruding the incubated HEK293 cell suspension through polycarbonate membranes having pore sizes of about 5 μm, about 0.8 μm, and about 0.4 μm to form nanoscale vesicles displaying the chimeric antigen receptor on an outer surface thereof and encapsulating the doxorubicin, and
recovering the formed nanoscale vesicles.

In one example, the incubating step comprises incubating the transfected HEK293 cells with doxorubicin at a concentration of about 10 μg/mL for a period of two hours at 37° C.

In one example, the recovered nanoscale vesicles have a mean particle size of from about 120 nm to about 160 nm as measured by nanoparticle tracking analysis.

In one example, the method comprising the step of removing unencapsulated doxorubicin (DOX) from the recovered nanoscale vesicles by ultracentrifugation for about 2 hours. In one example, the ultracentrifugation may be performed at 100,000×g for about 2 hours. In other examples, other processes to remove unencapsulated DOX may be used such as for example, tangential flow filtration or ultrafiltration.

In one example, the method comprising the step of formulating the recovered nanoscale vesicles into a pharmaceutical composition comprising a pharmaceutically acceptable carrier.

In accordance with a further aspect, the present disclosure provides an engineered exosome-mimetic vesicle for targeted drug delivery, comprising:
a biomimetic vesicle derived from a eukaryotic cell engineered to express a Chimeric Antigen Receptor (CAR) protein on its surface,
a therapeutic cargo encapsulated within the vesicle, and
wherein the CAR protein is configured to specifically recognize and bind to a target antigen on a target cell, and the vesicle is produced by a mechanical extrusion method.

In one example, the eukaryotic cell is a human embryonic kidney (HEK293) cell, wherein the CAR protein is a CD19-targeting CAR, and wherein the therapeutic cargo is doxorubicin (DOX).

The engineered exosome-vesicle is advantageous because it combines the targeting specificity of CAR-T cells with the drug delivery capacity of exosomes. The engineered exosome-mimetic vesicle is beneficial as it exhibits the physical characteristics similar to those of natural exosomes, as well as CAR-mediated targeting function and significant anti-tumor activity both in vitro and in vivo. The engineered exosome-mimetic vesicle advantageously reduces toxicities, complex manufacturing and poor penetration into solid tumors.

In one example, the vesicle has a peak particle size distribution of approximately 145 nm.

In one example, the vesicle exhibits sustained release kinetics of the therapeutic cargo.

In one example, the mechanical extrusion method involves sequentially extruding cells through polycarbonate porous membranes with progressively smaller pore sizes, and wherein the therapeutic cargo is encapsulated within the vesicle during its formation via the mechanical extrusion method.

In accordance with a further aspect, the present disclosure provides a pharmaceutical composition for treating a malignant tumor, comprising:
a therapeutically effective amount of the engineered exosome-mimetic vesicles according to any one of the statements above, and
a pharmaceutically acceptable excipient.

In one example, the malignant tumor is a lymphoma.

In one example, the target antigen is CD19.

In one example, the pharmaceutical composition is used in the treatment of cancer.

In one example, the engineered exosome-mimetic vesicles is administered intravenously.

The engineered exosome-mimetic vesicles are advantageous because they demonstrate enhanced tumor-specific drug accumulation and antitumor efficacy in vivo.

The described technology introduces several improvements, including a single-step mechanical extrusion process for engineered exosome-mimetic vesicle production, surface display of CAR proteins for targeted drug delivery in the engineered exosome-mimetic vesicle, biomimetic vesicle design, enhanced drug encapsulation and release kinetics, tumor-specific targeting and efficacy, and a versatile platform for off-the-shelf nanotherapeutics. These features collectively address the limitations in existing exosome-based and cell-based therapies, offering scalable, efficient, and clinically applicable solutions.

In accordance with a further aspect, the present disclosure provides a systematic platform for exosome-based targeted drug delivery, which includes a cell line, a plasmid design and transfection strategy, a specific method for exosome production, a purification and enrichment approach. In an example embodiment, the target tissues or organs are selected from: blood vessels, tumors, heart and brain.

In one example, the cell line is a common cell line, such as for example a eukaryotic cell that can be transfected.

In one example, the cell line includes production materials for artificial exosomes after transfection of specific plasmids.

In one example, the plasmids include a surface protein coding plasmid. For example, the surface protein coding plasmid isa transposase-coding plasmid for stable transfection.

In one example, the plasmids comprise vectors, and the vectors are pT3 and pT4, and the transposase is restricted to a Sleeping Beauty transposon system. In one example, the surface protein is a CAR protein, and the vectors are pT3 and pT4.

In one example, the transfection method is lipofectamine transfection or electroporation.

In one example, the exosomes are produced by extrusion of cells using a mini extruder or a scale up extruder.

In one example, the extrusion is a continuous extrusion process of extruding the cells through a filter membrane having progressively smaller pore sizes. The filter membrane for extrusion, for example, comprises pore sizes of 10 μm, 5 μm, 1 μm and 0.4 μm. The extrusion times may be 2˜10 times. Alternatively, the pore size may be 10 μm, 5 μm, 1 μm, 0.4 μm or 0.2 μm, or as in some examples, the porous membranes may include pores that are of a single or multiple (combination) of pore sizes, such as (10 μm, 5 μm, 1 μm, 0.4 μm), (5 μm, 1 μm, 0.4 μm, 0.2 μm), (5 μm, 0.8 μm, 0.2 μm) or any combination thereof. Extrusion times may also be in the range of 2˜30 times.

In one example, the molecules are wrapped into exosomes in the extrusion process as described. In an example, the molecules are restricted to biological molecules such as DNA, RNA, protein or chemicals such as anti-tumor drugs. The DNA may include but is not limited to plasmid DNA or DNA fragments. The RNA may include but is not limited to siRNA and mRNA. The drugs may include but are not limited to doxorubicin and cisplatin.

In one example, a purification method is performed after the extrusion process. In another example, the purification method includes ultracentrifugation and ultrafiltration.

In one example, the surface proteins of produced exosome-mimetic vesicles are targeted to tissue or organs such as blood vessels, tumors, the heart or the brain.

In one example, the routes of administration of the produced engineered exosome-mimetic vesicles include intravenous, inhalation or oral administration.

The term “comprising” (and its grammatical variations) as used herein are used in the inclusive sense of “having” or “including” and not in the sense of “consisting only of”.

The term “eukaryotic cell” as used herein is a cell characterized by a membrane bound nucleus that encloses the cell's genetic material. The eukaryotic cell may be a common cell that can be transfected such as for example HEK293, fibroblast, stem cells or other common cells.

It is to be understood that, if any prior art information is referred to herein, such reference does not constitute an admission that the information forms a part of the common general knowledge in the art.

### BRIEF DESCRIPTION OF THE DRAWINGS

Embodiments of the present invention will now be described, by way of example, with reference to the accompanying drawings in which:

FIG. 1 illustrates a flow chart of an example embodiment of a method of producing an engineered exosome-mimetic vesicle.

FIG. 2 illustrates a flow chart of a further embodiment of a method of producing an engineered exosome-mimetic vesicle.

FIG. 3 illustrates an example of an engineered exosome-mimetic vesicle.

FIG. 4 illustrates a schematic illustration of the production of engineered exosome-mimetic vesicles in accordance with an example embodiment.

FIG. 5 illustrates a plot of the size distribution of engineered exosome-vesicles (i.e., CAR-exosomes).

FIG. 6 illustrates transmission electron microscopy images (negative staining) of engineered exosome-mimetic vesicles.

FIG. 7 illustrates a fluorescent image of engineered exosome-mimetic vesicles.

FIG. 8 illustrates an example in vitro release profile of doxorubicin (DOX) over time.

FIG. 9 illustrates the results of the evaluation of the targeting specificity of CD19 CAR-exosomes.

FIG. 10 illustrates CD19 expression levels in Jurkat and Raji cells.

FIG. 10A illustrates validation of the targeting ability of engineered exosome-mimetic vesicles (i.e., CAR exosomes) on Jurkat and Raji cells.

FIG. 11 illustrates growth of Raji cells was inhibited following treatment with CAR-exosomes, in a manner dependent on the concentration of DOX used during extrusion.

FIG. 12 illustrates a plot that shows the evaluation of antitumor efficacy of the CAR-exosomes in vivo.

### DETAILED DESCRIPTION

Cell therapy, particularly chimeric antigen receptor T-cell (CAR-T) therapy, has revolutionized the treatment of malignant tumors. By genetically engineering T cells to specifically recognize tumor-associated antigens, CAR-T therapy has demonstrated remarkable efficacy in inducing tumor regression. However, its clinical application still faces multiple challenges, especially in the treatment of solid tumors. These challenges include severe and potentially life-threatening toxicities (such as cytokine release syndrome and immune effector cell-associated neurotoxicity syndrome), high treatment costs, limited accessibility due to complex manufacturing processes, and poor penetration into the tumor microenvironment.

There is a clinical need to develop novel therapeutic strategies that retain the precise targeting advantages of CAR-T cells while offering improved safety, broad application, and the potential for off-the-shelf use.

In recent years, extracellular vesicles, particularly exosomes, have emerged as a promising natural drug delivery platform. As endogenous nanovesicles, exosomes exhibit excellent biocompatibility, low immunogenicity, and the ability to cross biological barriers. Their lipid bilayer may be engineered to display targeting ligands, while their internal cavity can be loaded with various therapeutic payloads, including nucleic acids and small-molecule drugs. Exosomes derived from CAR-T cells carry intact CAR structures and demonstrate target specificity and cytotoxic activity similar to those of CAR-T cells. However, their production still relies on CAR-T cells, resulting in limited yield, high cost, and difficulties in meeting the demands of large-scale clinical applications.

The present disclosure relates to a method of producing an engineered exosome-mimetic vesicle. In particular, the method can be used to construct bioengineered vesicles possessing both CAR-mediated targeting function and potent cytotoxic capacity.

The present detailed description provides illustrative embodiments of the disclosed subject matter, which generally relates to the field of bioengineered therapeutic platforms, specifically to methods for producing chimeric antigen receptor (CAR)-presenting exosome-mimetic vesicles loaded with therapeutic agents. These vesicles combine the targeting precision of CAR-T cells with the drug delivery capabilities of exosomes, offering an innovative approach to addressing challenges in oncology, such as limited tumor penetration, elevated treatment costs, and significant toxicities associated with conventional therapies.

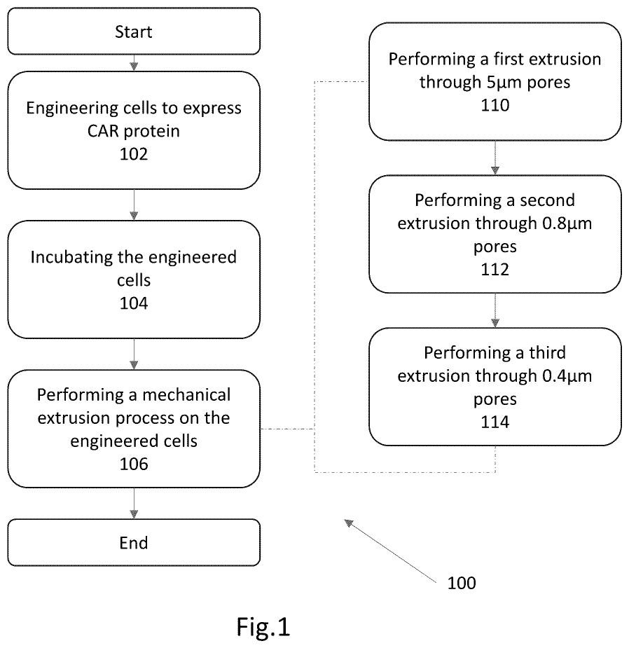

Referring to FIG. 1, there is shown an example method 100 for producing one or more engineered exosome-mimetic vesicles for targeted drug delivery. The method comprises the step 102. Step 102 includes engineering one or more eukaryotic cells to stably express a Chimeric Antigen Receptor (CAR) protein on their surface. Step 104 includes incubating the one or more engineered eukaryotic cells with a therapeutic cargo. Step 106 includes subjecting the one or more incubated cells to a mechanical extrusion process to form nanoscale one or more engineered exosome-mimetic vesicles with the CAR protein displayed on their surface and the therapeutic cargo or payload encapsulated within. The process 100 produces engineered exosome-mimetic vesicles that carry a drug as the therapeutic cargo.

In one example, the eukaryotic cells are human embryonic kidney (HEK293) cells. In one example, the CAR protein is a CD19-targeting CAR. The eukaryotic cells may be in the form of a cell line. The cell line can function as the production material for the engineered exosome-mimetic vesicles after transfection of specific surface protein. In one example, the therapeutic cargo is doxorubicin (DOX). Other drugs may be used as therapeutic cargo. In the described embodiment, DOX is used as the engineered exosome-mimetic vesicle cargo and is used to treat cancer e.g., Raji lymphoma.

In one example, the mechanical extrusion process comprises sequentially extruding the cells through polycarbonate porous membranes having progressively smaller pore sizes.

The method 100 as described is advantageous because it produces engineered exosome-mimetic vesicles (CAR-exosomes) which combine the targeting specificity of CAR-T cells with drug delivery capacity of exosomes. The method 100 provides high production efficiency. The method 100 is also advantageous because the drug e.g., DOX is loaded into the exosome-mimetic vesicle during vesicle formation, compared to post isolation loading. This is advantageous because it preserves vesicle integrity and improves drug encapsulation efficiency.

The mechanical extrusion process 106 comprises sequentially extruding the cells through polycarbonate porous membranes having progressively smaller pore sizes. In one example, the polycarbonate porous membranes comprise pore sizes of 5 μm, 0.8 μm, and 0.4 μm. In other examples, the pore size may be 10 μm, 5 μm, 1 μm, 0.4 μm or 0.2 μm, or as in some examples, the porous membranes may include pores that are of a single or multiple (combination) of pore sizes, such as (10 μm, 5 μm, 1 μm, 0.4 μm), (5 μm, 1 μm, 0.4 μm, 0.2 μm), (5 μm, 0.8 μm, 0.2 μm) or any combination thereof. Such combination of membrane pores may be determined based on the cell type, operation method and specific application.

The mechanical extrusion process 106 may comprise multiple extrusions 110, 112, 114. A first extrusion 110 is performed through a polycarbonate porous membrane having a pore size of 5 μm. A second extrusion 112 is performed through a polycarbonate porous membrane having a pore size of 0.8 μm. A third extrusion 114 is performed through a polycarbonate porous membrane having a pore size of 0.4 μm.

The method of producing the engineered exosome-mimetic vesicles may be a one-step extrusion process. Any suitable extrusion pressures can be used during the mechanical extrusion process. In one example, the extrusion process may be performed 2 to 10 times. Alternatively, extrusion times may also be in the range of 2˜30 times.

The extrusion process may be performed by a suitable extruder such as for example mini-extruder, scale-up extruder or any other suitable extruder.

The step of engineering the human embryonic kidney cells (HEK293) comprising performing plasmid transfection on the HEK293 cells to engineer the HK293 cells to express CD19-targeting CAR protein. The cells were prepared as a suspension (3×10⁶ cells/ml).

The transfection method may be any one of lipofectamine transfection or electroporation.

In one example, the incubating step 104 comprises incubating the transfected HEK293 cells with doxorubicin (DOX) at a concentration of about 10 μg/mL for a period of two hours at 37° C.

The method 100 optionally comprises the additional step of isolating the engineered exosome-mimetic vesicles. The engineered exosome-mimetic vesicles may be isolated by an appropriate isolation process e.g., density gradient ultracentrifugation or tangential flow filtration or any other suitable isolation process.

Optionally, the method 100 may comprise the additional step of removing unencapsulated DOX from the recovered nanoscale vesicles by ultracentrifugation. The ultracentrifugation may be performed for about two hours. In one example, the ultracentrifugation may be performed at 100,000×g for about two hours. In another example, other processes to remove unencapsulated DOX may be used such as for example, tangential flow filtration.

The engineered exosome-mimetic vesicles may have a mean particle size of from about 120 nm to about 160 nm as measured by nanoparticle tracking analysis. In one example embodiment, the engineered exosome-mimetic vesicles comprise a peak size of 145 nm.

The engineered exosome-mimetic vesicle as described may be configured to achieve a cumulative release concentration of 5.57 μg/ml at nine hours. The cells may be incubated with DOX for two hours.

The method 100 provides an improved biomimetic vesicle system that integrates the targeting specificity of CAR-T cells with the drug delivery capacity of exosomes. The method 100 provides a one-step mechanical extrusion process that extrudes engineered cells through progressively smaller pores. The method 100 generates engineered eukaryotic cells e.g., HEK293 cells stably expressing a CD19 targeting CAR to produce nanoscale vesicles loaded with DOX during formation.

The single-step method 100 combines vesicle formation and drug loading. Conventional methods involve separate steps for vesicle isolation and drug loading, which compromise efficiency and vesicle integrity. The method 100 provides a streamlined approach with enhanced scalability, reproducibility, and clinical applicability.

In alternative embodiments, the engineered exosome-vesicles may be loaded with other therapeutic payloads or cargo (i.e., drugs) to DOX. In alternative examples, the therapeutic cargo can be one of: etoposide, rituximab, cisplatin, paclitaxel, tyrosine kinase inhibitors (TKIs), bortezomib or any other suitable drug. The method 100 can be used to load another suitable cancer treatment drug or drugs into the engineered exosome-mimetic vesicle.

In one example, the HK293 cells can be transfected with CAR plasmids (such as anti-CD19, anti-FAP plasmids) to express the protein that can bind with CD19, FAP or other targeted proteins. By using one or more extruders to perform extrusion, large amounts of artificial exosomes can be produced using the transfected cell line, and molecules such as biological molecules (DNA, RNA, protein) and chemical drugs can be wrapped into the artificial exosomes during the extrusion process. The drugs may be DOX or other suitable drugs.

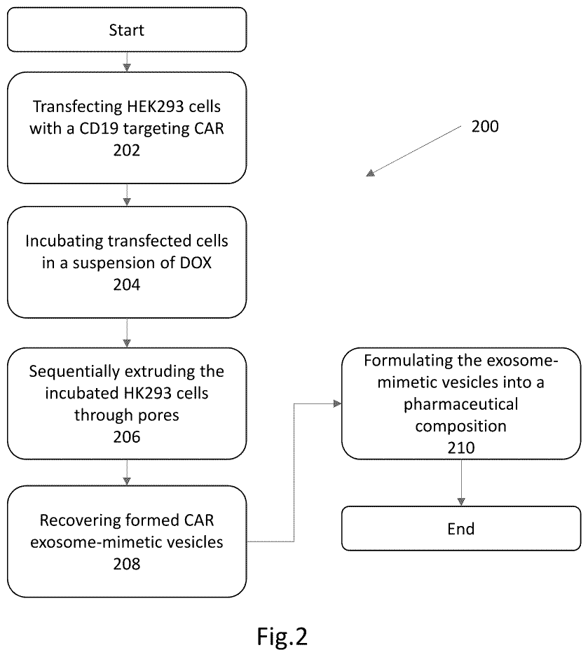

FIG. 2 illustrates a further embodiment of a method 200 of producing chimeric antigen receptor-presenting (CAR) exosome-mimetic vesicles loaded with a therapeutic agent. The method 200, comprises step 202. Step 202 includes stably transfecting human embryonic kidney (HEK293) cells with a nucleic acid encoding a CD19-targeting chimeric antigen receptor. Step 204 comprises incubating the transfected HEK293 cells in a suspension comprising doxorubicin (DOX). Step 206 includes sequentially extruding the incubated HEK293 cell suspension through polycarbonate membranes having pore sizes of about 5 μm, about 0.8 μm, and about 0.4 μm to form nanoscale vesicles displaying the chimeric antigen receptor on an outer surface thereof and encapsulating the DOX. Step 208 comprises recovering the formed nanoscale vesicles. The formed nanoscale vesicles are the CAR-exosome-mimetic vesicles.

In one example, the nucleic acid may comprise a plasmid vector encoding an extracellular single-chain variable fragment (scFv) specific for CD19.

The incubating step 204 may comprise incubating the transfected HEK293 cells with DOX at a concentration of about 10 μg/mL for a period of two hours at 37° C. Preferably, the nanoscale vesicles have a mean particle size of from about 120 nm to about 160 nm as measured by nanoparticle tracking analysis. In one example, the CAR-exosome-mimetic vesicles may comprise a size of 145 nm.

In one example, the method 200 may comprise the step of removing unencapsulated DOX from the recovered nanoscale vesicles by ultracentrifugation for about two hours. In one example, the ultracentrifugation may be performed at 100,000×g for about two hours. In other examples, other processes to remove unencapsulated DOX may be used, including tangential flow filtration.

The method 200 includes the optional step of formulating the recovered nanoscale vesicles 210 into a pharmaceutical composition comprising a pharmaceutically acceptable carrier. In an example embodiment, the pharmaceutical composition comprises the CAR-exosome-mimetic vesicles. The pharmaceutical composition may be injected into a patient to deliver drugs to a target site e.g., a tumor.

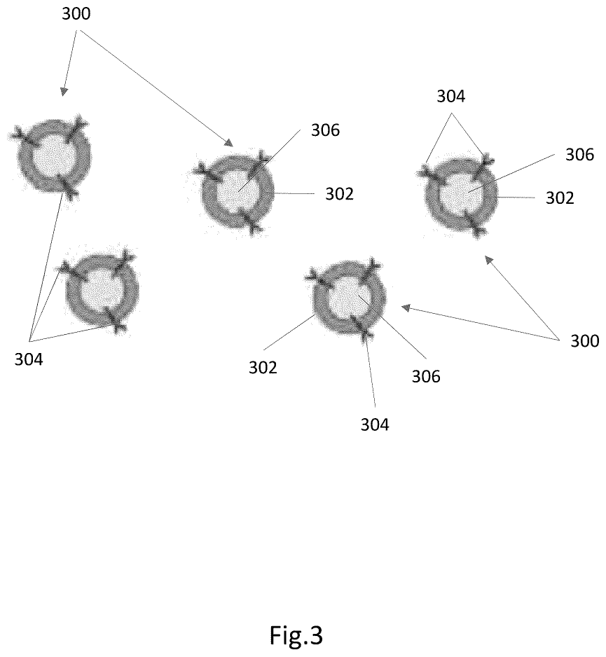

FIG. 3 illustrates an example embodiment of an engineered exosome-mimetic vesicles 300 for targeted drug delivery. FIG. 3 illustrates multiple vesicles 300 formed by method 100 or method 200. Each vesicle 300 comprises a biomimetic vesicle derived from a eukaryotic cell 302 engineered to express a Chimeric Antigen Receptor (CAR) protein 304 on its surface. The vesicle 300 includes a therapeutic cargo encapsulated 306 within the vesicle. The CAR protein is configured to specifically recognize and bind to a target antigen on a target cell, and the vesicle 300 is produced by a mechanical extrusion method.

In one example, the eukaryotic cell is a human embryonic kidney (HEK293) cell 302. In another example, the CAR protein 304 is a CD19-targeting CAR, and the therapeutic cargo 306 is DOX. The therapeutic cargo 306 may comprise an alternative therapeutic cargo (i.e., drugs) to DOX. The alternative therapeutic cargo 306 can be, for example, etoposide, rituximab, cisplatin, paclitaxel, tyrosine kinase inhibitors (TKIs), bortezomib or any other suitable drug.

In a most preferred embodiment, the vesicle 300 is in the nanometer (i.e., nanoscale) size range. For example, the engineered exosome-mimetic vesicle 300 is between 120 nm and 160 nm in size. In one example, the vesicle 300 has a peak particle size of approximately 145 nm. The vesicle 300 is formed to exhibit sustained release kinetics of the therapeutic cargo.

The vesicle 300 may be produced by the mechanical extrusion method 100 or 200 as described earlier. In one example, the mechanical extrusion method involves sequentially extruding cells through polycarbonate porous membranes with progressively smaller pore sizes, and wherein the therapeutic cargo is encapsulated within the vesicle during its formation via the mechanical extrusion method.

The CAR-exosomes 300 (i.e., engineered exosome-mimetic vesicles) formed by the method 100 or method 200 are naturally decorated with CAR structures on their surface, enabling specific targeting of tumors or cancer cells. The CAR-exosomes 300 are also loaded with a therapeutic cargo for simultaneous drug delivery. The engineered exosome-mimetic vesicles (CAR-exosomes 300) are multifunctional vesicles 300 that can specifically recognize and bind to target positive tumor cells, internalize into the cells and elicit potent cytotoxic effects, thereby mimicking the core functions of CAR-T cells while minimizing the associated risks.

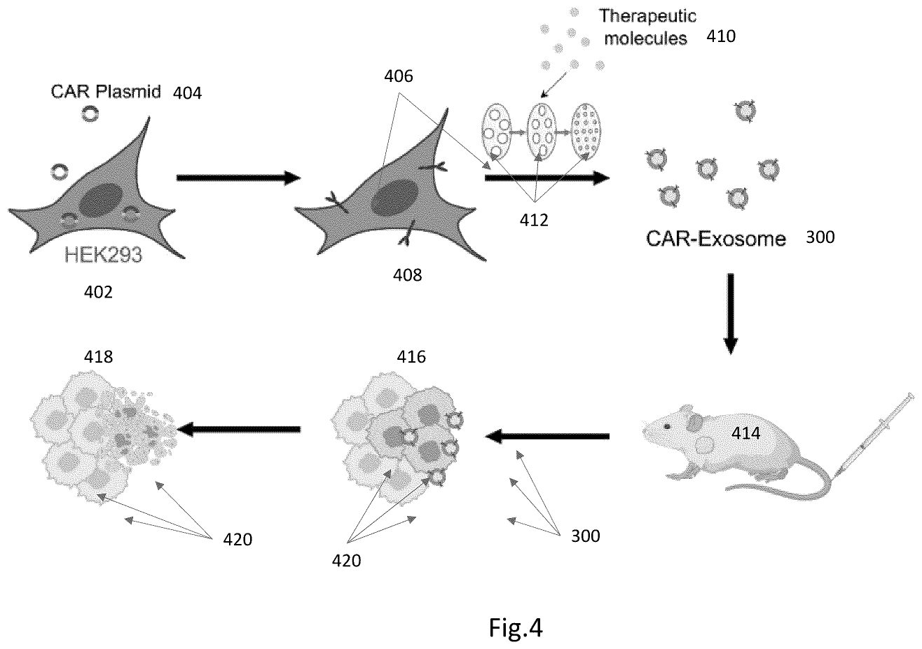

FIG. 4 illustrates a schematic of the production of the engineered exosome-mimetic vesicles 300 (i.e., CAR-exosomes). Referring to FIG. 4, HEK293 cells 402 are first transfected with CAR-encoding plasmid 404 to stably express the designed CAR protein 406 on their surface. Engineered cells 408 are produced and include a CAR protein on their surface. The HEK293 cells are an example of eukaryotic cells used to create the CAR-exosome. The engineered cells 408 may be subject to sequential extrusion through polycarbonate membranes 412 having progressively smaller pore sizes. Therapeutic molecules 410 e.g., DOX molecules 410 may be encapsulated into the formed exosome-mimetic vesicles 300. The therapeutic molecules may be encapsulated as therapeutic cargo within the vesicles 300. The drug molecules 410 may be introduced during the extrusion process and encapsulated into newly formed exosome-mimetic vesicles 300.

The resulting exosome-mimetic vesicles 300 (i.e., CAR-exosomes) can be administered intravenously. The exosome-mimetic vesicles 300 display CAR protein on the surface and are internally loaded with a drug e.g., DOX. As shown in FIG. 4, the vesicles 300 (i.e., CAR-exosomes) are administered in immunodeficient mice 414 bearing Raji lymphoma xenografts. The CAR-exosomes 300 demonstrated significant tumor killing efficacy and tumor growth inhibition at 416, 418 in FIG. 4. The tumor cells 420 are shown in FIG. 4, and step 418 illustrates the tumor cells 420 being killed by the CAR exosomes 300.

The CAR-exosomes 300 may be formulated as a pharmaceutical compound that may be injectable. Alternatively, the CAR-exosomes 300 may be formulated as a pharmaceutical composition for oral or inhalation delivery to a patient.

In one example, the exosome-mimetic vesicles (CAR-exosomes) may be formulated as a pharmaceutical composition for treating a malignant tumor. The pharmaceutical composition may comprise a therapeutically effective amount of the engineered exosome-mimetic vesicles as described earlier. The engineered exosome-mimetic vesicles may be the CAR-exosome 300 as described. The exosome-mimetic vesicles may also be formed by a method 100 or method 200 as described earlier. The pharmaceutical composition may comprise a pharmaceutically acceptable excipient. In one example, the target antigen may be CD19.

The pharmaceutical composition may be used in the treatment of cancer, in particular a malignant tumor. In one example, the malignant tumor may be a lymphoma. The engineered exosome-mimetic vesicles 300 may be administered intravenously. The pharmaceutical composition comprising the engineered exosome-mimetic vesicles 300 (CAR-exosomes) may be administered intravenously. The engineered exosome-mimetic vesicles 300 are advantageous because they demonstrate enhanced tumor-specific drug accumulation and antitumor efficacy in vivo.

The produced engineered exosome-mimetic vesicles 300 were tested to determine physical properties and performance in vitro and in vivo.

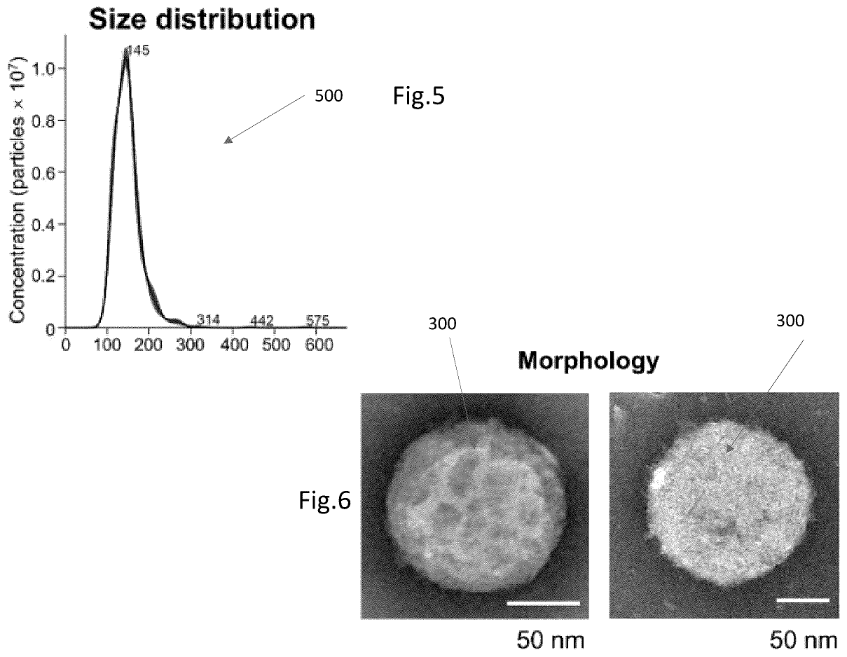

FIGS. 5 to 8 illustrate results of the characterization of the engineered exosome-vesicles 300 including characterizing size, morphology, drug encapsulation and drug release. FIG. 5 illustrates a plot 500 of the size distribution of the engineered exosome-vesicles 300 (i.e., CAR-exosomes). The plot 500 may be created by nanoparticle tracking analysis (NTA). NTA revealed that the prepared engineered exosome-vesicles 300 (i.e., CAR-exosomes) exhibited peak particle size distribution at 145 nm. This falls within the typical size range of natural exosomes.

FIG. 6 illustrates images of the morphological features of the engineered exosome-mimetic vesicles 300. FIG. 6 illustrates transmission electron microscopy images (negative staining) of the engineered exosome-mimetic vesicles 300. As shown in FIG. 6, the morphological features of the CAR-exosomes 300 are consistent with natural exosomes. The diameters of the vesicles 300 are consistent with classical exosome characteristics. The microscope images indicate the CAR-exosomes 300 closely resemble natural exosomes in terms of physical properties.

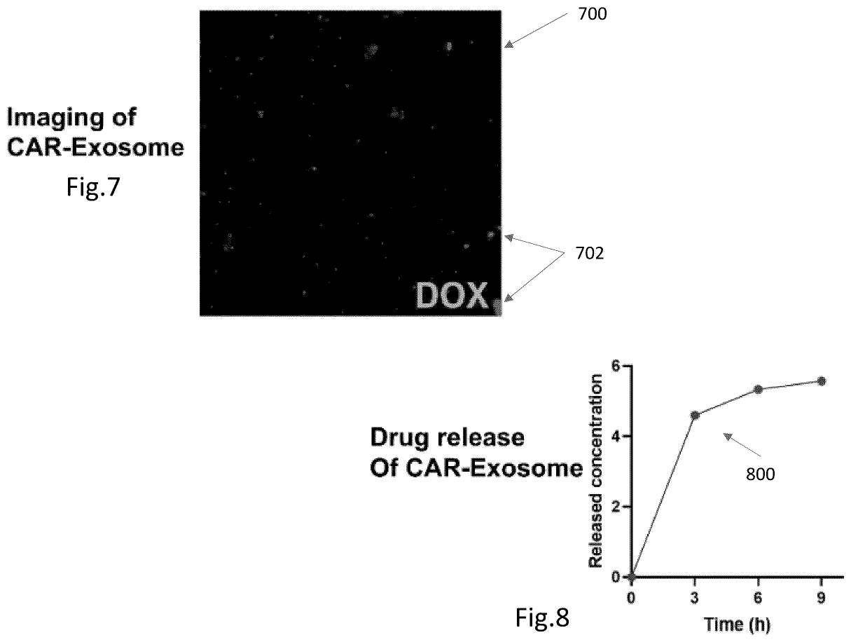

FIG. 7 illustrates a fluorescent image 700 of the engineered exosome-mimetic vesicle (or vesicles) 300. The fluorescent image or images may be captured by an appropriate camera or image capture device e.g. a fluorescence microscope. DOX has an inherent fluorescence. The fluorescent image 700 captured by fluorescence microscopy illustrates the fluorescent blobs or fluorescent points 702. These indicate successful DOX loading into the engineered exosome-mimetic vesicles 300.

FIG. 8 illustrates an example in vitro release profile 800 of DOX over time. The drug release experiments show an initial rapid release within the first three hours, which slows after six hours as shown in the in vitro release profile 800. The first section of the graph 800 illustrates a rapid release of DOX drug over the first three hours. The rate of release slows between three hours and six hours. The drug release slows down significantly after six hours as compared to the release profile in the first three hours. A cumulative release concentration of 5.57 μg/mL at nine hours, as shown in the release profile 800 of FIG. 8.

In vitro targeting capability of the engineered exosome-mimetic vesicles 300 (CAR-exosomes 300) was tested. To evaluate the in vitro targeting capability of CAR-exosomes 300, CD19 positive Raji cells were co-cultured with CD19 negative Jurkat cells. CD19 targeted CAR-exosomes 300 were introduced to the co-cultured cells. After two hours of incubation, fluorescence signals of DOX and CD19 were detected under excitation wavelengths of 488 nm and 647 nm, respectively using confocal microscopy.

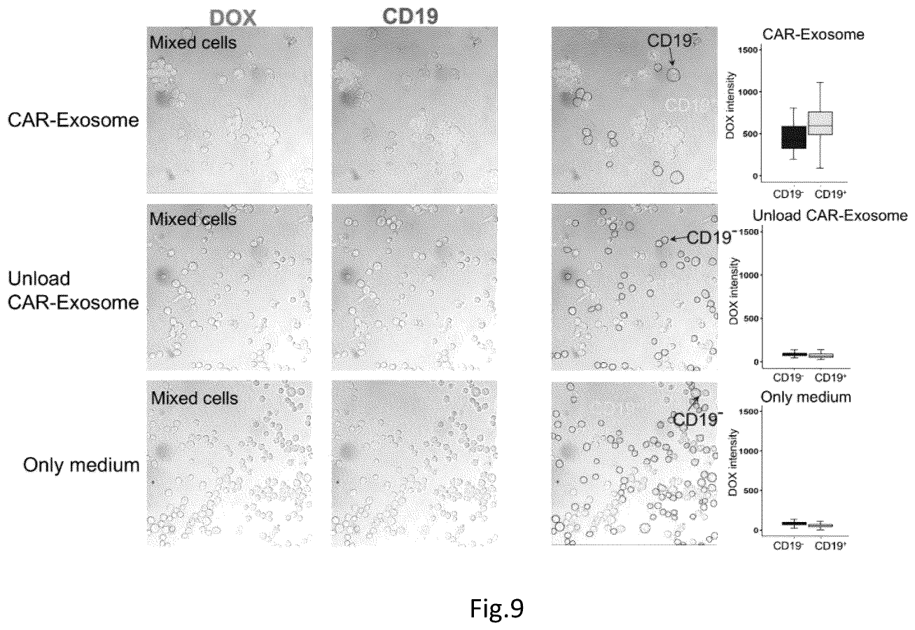

FIG. 9 illustrates the targeting specificity of the CD19 CAR-Exosomes. A co-culture system of CD19-positive (Raji, CD19+) and CD19-negative (Jurkat, CD19−) cells was established and treated with the engineered exosomes. Referring to FIG. 9, the results show that cells exhibiting distinct magenta fluorescence (i.e., the DOX) also displayed CD19 positive signals (red). This indicates that the uptake efficiency of CAR-exosomes 300 was higher in CD19⁺ Raji cells compared to CD19⁻ Jurkat cells. No DOX signal was detected in the control group treat with drug free CAR-exosomes. This further confirms that the observed fluorescence as shown in FIG. 9 originated from the specifically delivered drug, in this example DOX. The findings, as shown in FIG. 9, demonstrate that CAR-exosomes 300 (i.e., the engineered exosome-mimetic vesicles 300) can effectively target CD19-positive (i.e., CD19⁺) cancer cells in vitro.

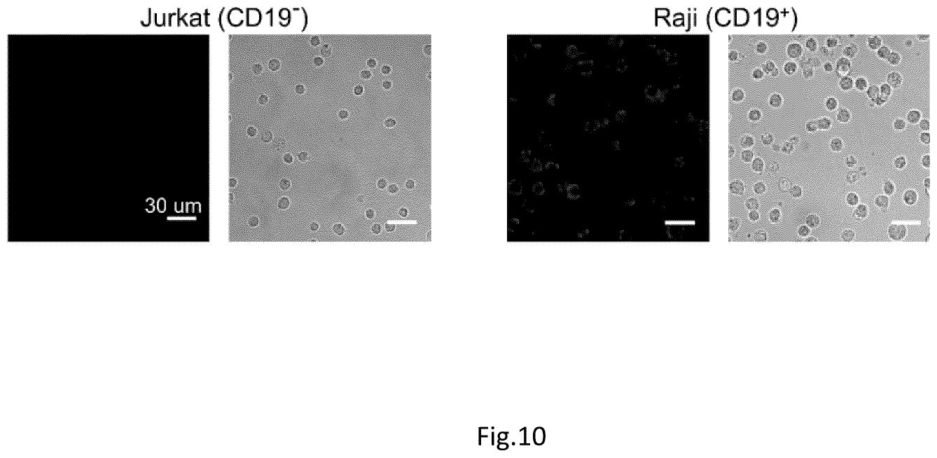

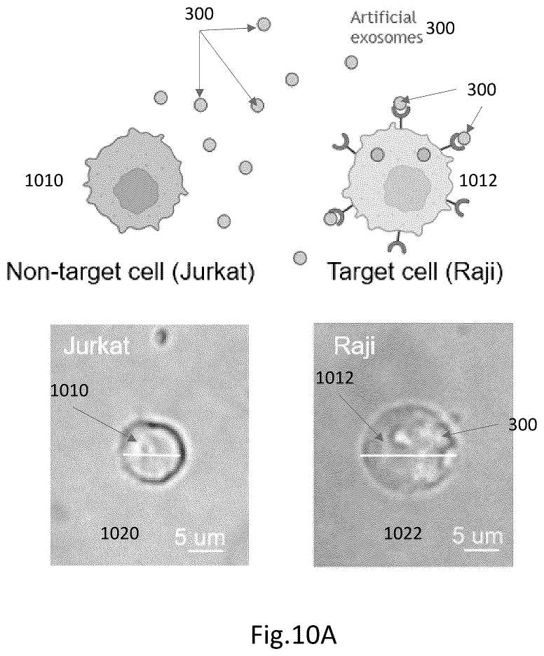

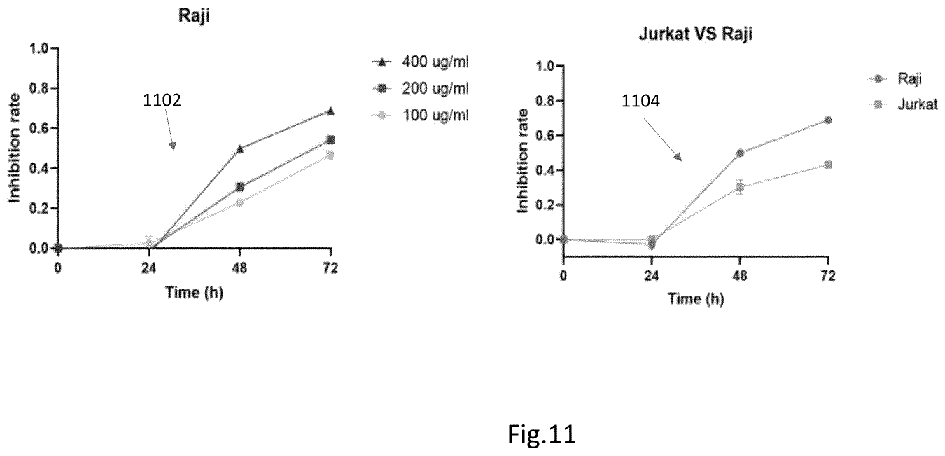

The cytotoxic effects of the engineered exosome-mimetic vesicles 300 (CAR-exosomes 300) were evaluated in both the Jurkat and Raji cells (i.e., evaluation of the in vitro killing ability). FIG. 10 and FIG. 11 illustrate the results of an evaluation of the killing ability of the developed engineered exosome-mimetic vesicles 300 (i.e., CAR-exosomes 300). FIG. 10 illustrates the CD19 expression levels in Jurkat and Raji cells. As shown in FIG. 10, Jurkat cells are CD19 negative, whereas Raji cells highly express CD19. After co-culturing the cells with the CAR-exosomes 300 loaded with different concentrations of DOX, cell proliferation was assessed using the CCK-8 assay method.

FIG. 10A illustrates a representation of the targeting ability of the CAR exosomes, as well a microscope images of the attached exosomes to the target cells. FIG. 10A illustrates non target Jurkat cells 1010, and the CAR exosomes 300 do not attach to the Jurkat cells 1010. As shown in FIG. 10A, the CAR exosomes 300 attach to the target Raji cells 1012. The CAR exosomes 300 attach to the receptors of the Raji cells 1012. Image 1020 illustrates the Jurkat cells 1010 with no real attachment of CAR exosomes 300. Image 1022 illustrates the Raji cells 1012 with several CAR exosomes 300 attached to it. This illustrates and validates the targeting ability of the engineered exosome-mimetic vesicles 300 (i.e., CAR exosomes) produced by the methods described herein.

FIG. 11 illustrates growth of Raji cells 1012 was inhibited following treatment with CAR-exosomes 300, in a manner dependent on the concentration of DOX used during extrusion. As shown in FIG. 11, the CAR-Exosomes 300 inhibited the proliferation of Raji cells 1012 in a concentration-dependent manner, as shown in graph 1102. Each line represents a different concentration. As the concentration of DOX increases, the inhibition rate increases. After 72 hours of treatment at a concentration of 400 μg/mL, the inhibition rate reached 70%, as shown in graph 1102. In contrast, the inhibitory effect of CAR-Exosomes 300 on Jurkat cells 1010 was significantly weaker, with much lower inhibition rates at both 48 h and 72 h time points compared to those in Raji cells 1012 indicating that the cytotoxic effect is CD19-dependent, as shown in graph 1104.

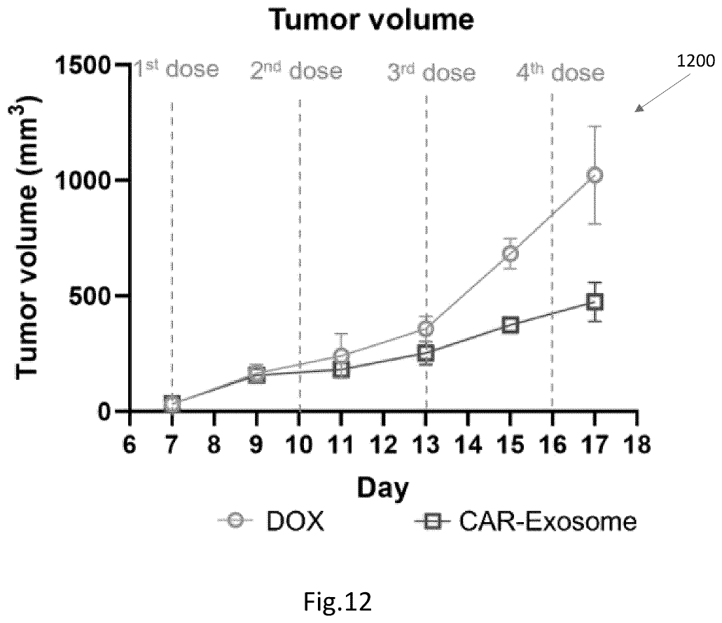

The inventors conducted an in vivo antitumor efficacy assessment was conducted. The anti-tumor efficacy of CAR-exosomes 300 in vivo was evaluated using a subcutaneous lymphoma xenograft mouse model. The changes in tumor volume are illustrated in FIG. 12. FIG. 12 illustrates a plot 1200 that shows the evaluation of antitumor efficacy of the CAR-exosomes 300 in vivo. Tumors in the free DOX control group (3 μg/g DOX) grew rapidly. In contrast, treatment with CAR-Exosomes 300 (loaded with 3 μg/g DOX) resulted in significant tumor regression. These results indicate that encapsulating DOX into targeted CAR-Exosomes 300 effectively enhances drug accumulation at the tumor site, thereby significantly improving its anti-tumor efficacy.

The described methods 100, 200 each are an improved preparation method for producing a biomimetic vesicle system 300 (i.e., a CAR-exosome). The CAR-exosome produced by the one step extrusion method is advantageous because it combines the targeting specificity of CAR-T cells with the drug delivery capacity of exosomes. The described methods of producing the engineered exosome-mimetic vesicles 300 is advantageous because it achieves high production efficiency through single step extrusion. The test results demonstrate the engineered exosome-mimetic vesicles 300 exhibit physical characteristics similar to those of natural exosomes, as well as CAR mediated targeting function and significant anti-tumor activity both in vitro and in vivo.

Marked tumor regression observed in xenograft models indicate the potential for the engineered exosome-mimetic vesicles (CAR-exosomes 300) to be developed into an effective treatment modality. The CAR-exosomes 300 as described herein overcome several of the limitations of conventional CAR-T therapy. The CAR-exosomes 300 described herein provide reduced toxicity, improved penetration into solid tumors and are produced by a simple one step mechanical extrusion process. Moreover, loading therapeutic drugs during vesicle formation—compared to post-isolation loading—offers notable advantages by preserving vesicle integrity and improving drug encapsulation efficiency.

The enhanced targeted delivery and anti-tumor efficacy achieved by CAR-exosomes 300 validate the effectiveness of the integrated production approach applied in methods 100, 200. Simultaneous extrusion and drug loading streamlines the manufacturing process and provides improved consistency between cargo loading and surface functionalization. This is an advantage for clinical scalability and production reproducibility.

The method of producing an engineered exosome-mimetic vesicle 100, 200 can be further extended to express various targeting molecules ((e.g., scFvs, nanobodies, or ligands) and deliver diverse cargoes including RNAs, proteins, or immunomodulators.

The methods 100, 200 provide a robust and scalable method for producing targeted therapeutic vesicles that mimic CAR-T cell functionality while circumventing its drawbacks. The engineered exosome-mimetic vesicles 300 (CAR-exosomes) can be used in oncology and beyond. The CAR-exosomes 300 provide a new class of hybrid nanotherapeutics and the methods 100, 200 provide a simple, robust and scalable method to produce the CAR-exosomes 300.

The described methods 100, 200 are advantageous as a single-step mechanical extrusion method for producing exosome-mimetic vesicles from engineered HEK293 cells stably expressing CAR proteins, with simultaneous drug encapsulation during vesicle formation. Conventional methods involve separate steps for vesicle isolation and drug loading, which compromise efficiency and vesicle integrity. The methods 100, 200 combine vesicle formation and drug loading into a single process. This approach is advantageous because it is streamlined, enhances scalability, reproducibility and clinical applicability.

The methods 100, 200 are also advantageous because the simultaneous extrusion and drug loading process also achieves high encapsulation efficiency and controlled, sustained release of therapeutic cargo. This improves therapeutic efficacy and reduces off-target toxicity.

The CAR-exosomes 300 described herein are advantageous because they exhibit size, morphology, and membrane characteristics closely resembling natural exosomes. The CAR-exosomes 300 are advantageous due to enhanced biocompatibility, reduced immunogenicity and improved clinical utility.

The CAR-exosomes 300 may be used on any suitable target tissues or organs, such as for example, blood vessels or tumors or the heart or brain. The CAR-exosomes 300 may be used to deliver medication to any target tissue.

The method 100, 200 as described enables rapid adaptation to different targeting molecules and therapeutic cargoes, supporting universal, off-the-shelf nanotherapeutics for oncology. The scalability and adaptability of the method 100, 200 is beneficial, as existing cell-based therapies are patient-specific, costly, and challenging to scale. This approach eliminates the need for patient-specific cell sourcing, providing a broadly applicable solution for various therapeutic uses.

A further example of an efficient and productive platform to produce engineered exosomes for targeted drug delivery to tissues including but not limited the heart will be described. In the platform, HEK293 cells play an important role in the manufacture of small exosome-like vesicles in a cost- and time-effective way, according to customized demands. HEK293 cells are transfected with CAR plasmids (such as anti-CD19, anti-FAP plasmids) to express the protein that can bind with CD19, FAP or other targeted proteins. By taking advantage of extruders (mini-extruder, scale-up extruder), large amounts of artificial exosomes can be produced using the transfected cell line, and molecules such as biological molecules (DNA, RNA, protein) and chemical drugs can be wrapped into the artificial exosomes during the extrusion process. After washing and purification of the obtained exosomes, characterization and imaging procedures are performed for quality control.

The exosome products may be used for targeted delivery of drugs (such as Doxorubicin) to cells with selected surface marker. Exosomes can enter the body through intravenous injection and travel in the circulating systems. The surface CAR protein will mediate the targeted delivery of drugs to specific cells, and there will be less uptake of exosomes than traditional lipid nanoparticles in liver, kidney or other tissues. As a result, the therapeutic efficiency will be dramatically improved, and the side effects will be decreased due to less off-target effects.

In one example, there is disclosed a systematic platform for exosome-based targeted drug delivery, which includes a cell line, a plasmid design and transfection strategy, a specific method for exosome production, a purification and enrichment approach. The target tissues or organs can be blood vessels, tumors, heart and brain.

The platform may include a common cell line that can be transfected, such as HEK293, fibroblast, stem cells and common cancer cell lines. The cell line as recited which are the production materials for artificial exosomes after transfection of specific plasmids. The plasmids as recited, which include a surface protein-coding plasmid a transposase-coding plasmid for stable transfection. The vectors for the plasmids are pT3 and pT4, and the transposase is restricted to Sleeping Beauty transposon system. The transfection method is lipofectamine transfection or electroporation. The exosomes are produced by extrusion of cells using a mini-extruder or a scale-up extruder.

In one example, the pore sizes of filtration membrane for extrusion recited in example 6 are 10 μm, 5 μm and 1 μm and 0.4 μm. The extrusion times may be 2˜10. The molecules can be wrapped into exosomes in extrusion process are restricted to biological molecules such as DNA, RNA, protein, and chemicals such as anti-tumor drugs. The DNA includes but is not limited to plasmid DNA or DNA fragments, the RNA includes but is not limited to siRNA and mRNA, and the drugs includes but is not limited to doxorubicin and cisplatin. The purification methods after extrusion incudes ultracentrifugation and ultrafiltration. The surface proteins of produced exosomes may be targeted to tissue or organs such as blood vessels, tumors, heart and brain. The administration methods of the produced exosomes may include intravenous, inhalation or oral routes of administration.

The present disclosure describes examples of an efficient and productive platform for artificial exosome production, which can be used to carry therapeutical molecules to targeted tissue or organs. The production method can be applied to cells, tissues and organs. The method provides an efficient approach to manufacture engineered exosomes at low cost with a high yield. The establishment of a stably transfected cell line can improve the targeting efficiency and lower the off-target side effects. The invention provides a versatile platform for targeted drug delivery. The cell lines or surface markers can be adapted for different purposes.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

The description is provided for illustrative purposes only and is not intended to limit the scope of the described subject matter. Certain widely recognized principles, techniques, and components commonly employed in the field of bioengineering and nanomedicine may not be described in comprehensive detail to avoid obscuring the described subject matter. Additionally, various modifications, substitutions, and rearrangements of the described embodiments may be made without departing from the spirit and scope of the subject matter, as defined by the claims.

1. A method for producing one or more engineered exosome-mimetic vesicles for targeted drug delivery, comprising:
engineering one or more eukaryotic cells to stably express a Chimeric Antigen Receptor (CAR) protein on their surface;
incubating the one or more engineered eukaryotic cells with a therapeutic cargo; and
subjecting the one or more incubated cells to a mechanical extrusion process to form nanoscale one or more engineered exosome-mimetic vesicle with the CAR protein displayed on their surface and the therapeutic cargo encapsulated within.

2. The method of claim 1, wherein the eukaryotic cells are human embryonic kidney (HEK293) cells.

3. The method of claim 1, wherein the CAR protein is a CD19-targeting CAR.

4. The method of claim 1, wherein the therapeutic cargo is doxorubicin (DOX).

5. The method of claim 1, wherein the mechanical extrusion process comprises sequentially extruding the cells through polycarbonate porous membranes having progressively smaller pore sizes.

6. The method of claim 5, wherein the polycarbonate porous membranes comprise pore sizes of 10 μm, 5 μm, ‘μm, 0.8 μm, 0.4 μm, 0.2 μm or any combination thereof.

7. The method of claim 5, wherein a first extrusion is performed through a polycarbonate porous membrane having a pore size of 5 μm, a second extrusion is performed through a polycarbonate porous membrane having a pore size of 0.8 μm, and a third extrusion is performed through a polycarbonate porous membrane having a pore size of 0.4 μm.

8. The method of claim 1 wherein the step of engineering the human embryonic kidney cells (HEK293) comprising the step of performing plasmid transfection on the HEK293 cells to engineer the HK293 cells to express CD19-targeting CAR protein.

9. The method of claim 1, further comprising, the step of isolating the one or more engineered exosome-mimetic vesicles.

10. The method of claim 1, wherein the engineered exosomes comprise a peak size of 145 nm.

11. The method of claim 1, wherein the engineered exosome is configured to achieve a cumulative release concentration of 5.57 μg/ml at 9 hours.

12. The method of claim 3, wherein the cells are incubated with DOX for two hours.

13. A method of producing chimeric antigen receptor-presenting exosome-mimetic vesicles loaded with a therapeutic agent, comprising:
stably transfecting human embryonic kidney (HEK293) cells with a nucleic acid encoding a CD19-targeting chimeric antigen receptor;
incubating the transfected HEK293 cells in a suspension comprising doxorubicin (DOX);
sequentially extruding the incubated HEK293 cell suspension through polycarbonate membranes having pore sizes of about 5 μm, about 0.8 μm, and about 0.4 μm to form nanoscale vesicles displaying the chimeric antigen receptor on an outer surface thereof and encapsulating the doxorubicin, and
recovering the formed nanoscale vesicles.

14. The method of claim 13, wherein the incubating step comprises incubating the transfected HEK293 cells with doxorubicin at a concentration of about 10 μg/mL for a period of two hours at 37° C.

15. The method of claim 14, wherein the recovered nanoscale vesicles have a mean particle size of from about 120 nm to about 160 nm as measured by nanoparticle tracking analysis.

16. The method of claim 14, further comprising formulating the recovered nanoscale vesicles into a pharmaceutical composition comprising a pharmaceutically acceptable carrier.

17. An engineered exosome-mimetic vesicle for targeted drug delivery, comprising:
a biomimetic vesicle derived from a eukaryotic cell engineered to express a Chimeric Antigen Receptor (CAR) protein on its surface,
a therapeutic cargo encapsulated within the vesicle, and
wherein the CAR protein is configured to specifically recognize and bind to a target antigen on a target cell, and the vesicle is produced by a mechanical extrusion method.

18. The engineered exosome-mimetic vesicle of claim 17, wherein the eukaryotic cell is a human embryonic kidney (HEK293) cell, wherein the CAR protein is a CD19-targeting CAR, and wherein the therapeutic cargo is doxorubicin (DOX).

19. The engineered exosome-mimetic vesicle of claim 17, wherein the vesicle has a peak particle size distribution of approximately 145 nm.

20. The engineered exosome-mimetic vesicle of claim 17, wherein the mechanical extrusion method involves sequentially extruding cells through polycarbonate porous membranes with progressively smaller pore sizes, and wherein the therapeutic cargo is encapsulated within the vesicle during its formation via the mechanical extrusion method.
